(19) United States
(12) Patent Application Publication
(10) Pub. No.: US 2011/0086108 A1
(43) Pub. Date: Apr. 14, 2011

(54) Dissolving powders that can be mixed with water and used as an oral rinse

(76) Inventor: Bartholomew Weldon, Chicago, IL (US)

(21) Appl. No.: 12/605,455

(22) Filed: Oct. 26, 2009

Related U.S. Application Data
(60) Provisional application No. 61/250,604, filed on Oct. 12, 2009.

(51) Int. Cl. A61K 33/42; A61P 29/00

(52) U.S. Cl. 424/602

(57) ABSTRACT

This invention involves the formulation of stable powders which, when dissolved in water form a non-pressurized carbonated solution supersaturated with calcium and phosphate ions, and also containing the presence of carbon dioxide and sodium bicarbonate. The resulting solution is used as an oral rinse for the prevention and treatment of inflammatory processes of the soft tissues of the mouth, throat and oral cavity, which may result from infection or trauma to the oral mucosal tissue.

### RELATED US PATENT APPLICATION

Provisional Application Number: 61/250604

Filed: Oct. 12, 2009

### REFERENCES CITED

US Patent Documents:
U.S. Pat. No. 5,268,267; Tung; Dec. 7, 1993
Methods and compositions for mineralizing and fluoridating calcified tissues
U.S. Pat. No. 5,427,768; Tung; Jun. 27, 1995
Carbonated Solutions for treating, mineralizing and fluoridating calcified tissues and methods for their use
U.S. Pat. No. 5,993,785; Johansen, et al; Nov. 30, 1999
Mouthwash Compositions: Aqueous Solutions are disclosed which are supersaturated with respect to calcium phosphate(s)
U.S. Pat. No. 6,387,352; Johansen, et al; May 14, 2002
Mouthwash Compositions: Aqueous Solutions are disclosed which are supersaturated with respect to calcium phosphate(s)

### OTHER REFERENCES

Papas AS et al, “A prospective, randomized trial for the prevention of mucositis in patients undergoing hematopoietic stem cell transplantation”
Bone Marrow Transplantation (2003), 31: 705-12

Carl & Emrich, “Management of oral mucositis during local radiation and systemic chemotherapy: A study of 98 patients”
J Prosthet Dent (1991), 66: 361-69
Corral, LG et al, “Antimicrobial activity of sodium bicarbonate”
J Food Science (1988) 53; 3: 981-982

### BACKGROUND OF THE INVENTION

1. Field of the Invention:

This invention relates to calcium, phosphate and sodium bicarbonate compositions, for use as a mouthwash or oral rinse, for the prevention and treatment of inflammatory processes of the soft tissues of the mouth, throat and oral cavity.

2. Background of the Invention:

Human saliva plays a number of roles in the oral cavity, including aiding in the prevention and healing of infections in the mouth and the remineralization of teeth. Among the components found within human saliva are high concentrations of calcium and phosphate. It is believed that calcium plays a role in preventing oral infection, protecting the oral mucosa and healing wounds in the mouth. Phosphate helps to modulate pH balance in the mouth, protecting the teeth and repairing mucosal damage.

U.S. Pat. No. 5,268,167 discloses the use of carbonated calcium and phosphate solutions under pressure for mineralizing and fluoridating calcified tissues. The carbonated solution under pressurized carbon dioxide allowed for maintenance of pH and provided a stabilized atmosphere for the calcium phosphate solution. No mention is made of the use of this formulation for the treatment of soft tissues within the oral cavity.

U.S. Pat. No. 5,427,768 disclosed a solid powder mixture of calcium and phosphate, with the addition carbonate salts, that allows for the creation of a non-pressurized, carbonated calcium and phosphate solution which could be used as a mouth rinse to treat, mineralize and fluoridate calcified tissues. The inclusion of carbonate salts allows for control of pH, deposition of calcium phosphate and rapid formation of apatite upon dental tissue. No mention is made of the use of this formulation for the treatment of soft tissues within the oral cavity.

U.S. Pat. No. 5,993,785 discloses a method for the treatment and prevention of conditions of the soft tissue of the oral cavity comprising separate calcium and phosphate stock solutions which, when combined, form a supersaturated calcium phosphate formulation intended to be used as an oral rinse. This formulation would also include a stabilizing agent to maintain supersaturation of calcium phosphates for a period following mixing of agents and maintain a pH range of 5-8. The stabilizing agents specified should comprise one or more salts of innocuous ions. This patent contains no references to the inclusion of carbonate salts in its formulation and specifies that the resulting, supersaturated solution should be substantially free of carbon dioxide and acid salts, like sodium bicarbonate.

U.S. Pat. No. 6,387,352 discloses a method for remineralization of calcified, dental tissues without the need for carbonation, comprising separate calcium and phosphate stock solutions which, when combined, form a supersaturated calcium phosphate formulation intended to be used as an oral rinse. This formulation would also include a stabilizing agent to maintain supersaturation of calcium phosphates for a period following mixing of agents and maintain a pH range of 5-8. This patent contains no references to the inclusion of carbonate salts in its formulation and specifies that the resulting, supersaturated solution should be substantially free of carbon dioxide and acid salts, like sodium bicarbonate.

### BRIEF SUMMARY OF THE INVENTION

The present invention relates to the mixture of solid powders which, when dissolved into water form a non-pressurized, carbonated solution, supersaturated with calcium and phosphate ions and containing the presence of carbon dioxide and sodium bicarbonate.

It has been demonstrated that an oral rinse supersaturated with calcium and phosphate is effective in reducing the duration and severity of oral mucositis in patients receiving hematopoietic stem cell transplantation (Bone Marrow Transplantation (2003) 31: 705-12). In addition, sodium bicarbonate is recognized as having the beneficial effect of reducing acidity of oral fluids, diluting accumulating mucus in the oral cavity and discouraging yeast and bacterial colonization.

The advantage of this invention is that it meets several criteria for the “ideal” oral rinse in compromised patients: it reduces harmful oral microflora, promotes reepitheliazation of the soft tissue, it normalizes the PH of oral fluids, has an acceptable taste and is non-toxic (J Prosthet Dent (1991) 66: 361-69). In addition, the powder formation provides a more convenient and portable formulation than offered by carrying or stocking separate calcium and phosphate stock solutions, needed for mixing together immediately prior to use.

The present invention stays stable in dry powder form until dissolved in water to create a solution supersaturated with calcium phosphate(s), containing carbon dioxide and sodium bicarbonate, and having a pH within the range of 5-8.

### DETAILED DESCRIPTION OF THE INVENTION

The present invention provides the compositions and methods that can be used to treat inflammatory processes of the soft tissues of the mouth, throat and oral cavity. The preferred formulation of this solid powder mix contains a stable mixture of calcium salts, phosphate salts, and carbonate salts, particularly sodium bicarbonate. When water is added to these powders, the solids will dissolve rapidly to create a solution supersaturated with respect to calcium and phosphate ions and also containing the presence of carbon dioxide and sodium bicarbonate.

It is known that human saliva is normally supersaturated with respect to calcium and phosphate. The oral rinse created by the powders in this device should contain significantly higher concentrations of calcium and phosphate ions than normally found in saliva. Since the degree of supersaturation of human saliva can vary between individuals, it is not possible to state definitively what that level of supersaturation of the present invention should be. However, in many cases, the resulting solution may be supersaturated with calcium and phosphate ions in the order of 5 to 10 times those found in normal, human saliva.

The preferred formulation of this powder mix creates a solution supersaturated with respect to calcium and phosphate ions. Preferably, this supersaturated solution has a pH from about 5.0 to around 8.0, more preferably from about 6.0-7.5, similar to that of normal saliva.

Preferably, the concentration of calcium ions (total free and complexed) in the supersaturated solution is in the range of from 2 to about 40 mM, more preferably in the range of 4.5 to 16 mM.

Preferably the concentration of phosphate ions (total phosphate) in the supersaturated solution is in the range from about 0.5 to about 32 mM, more preferable in the range of 1 to 20 mM.

When properly formulated, the resulting carbonated calcium phosphate solution can provide many of the natural protections of human saliva. The solution can also include other beneficial ions such as fluoride and may also contain a localized analgesic agent such as benzocaine or lidocaine to palliate pain arising from inflammation of the soft tissues of the mouth, throat or oral cavity, or from oral wounds to the soft tissues of the mouth, throat or oral cavity.

The following examples serve to illustrate the composition and method of this invention, but are in no way limiting thereto:

### EXAMPLES

### Example 1

A solid powder containing a mixture of calcium salts, phosphate salts and sodium bicarbonate, which when mixed with water creates a solution supersaturated with calcium ions in the range of 4.5 to 16 mM, and phosphate ions in the range of 1 to 20 mM, and having a pH in the range of 6.0-7.5.

### Example 2

The procedure of Example 1 is repeated, except that the supersaturated solution also contains 5 mM fluoride ions.

### Example 3

The procedure of Example 1 is repeated, except that the solid mixture also contains solid powder of lidocaine, benzocaine, or other local, analgesic powder.

### Example 4

The procedure of Example 1 is repeated, except that solid mixture also contains solid powder of acid, such as citric or tartaric acid.

What I claim is:

1. A method for the prevention and/or treatment of inflammatory processes of the soft tissues of the mouth, throat and oral cavity, comprising:
(a) A mixture of stable powders containing calcium salts, phosphate salts, and carbonate salts, particularly sodium bicarbonate, intended to be dissolved in water to form a non-pressurized carbonated solution, containing sufficient carbonate and highly concentrated with calcium ions and phosphate ions, to create a mixed solution supersaturated with calcium phosphate and containing the presence of carbon dioxide and sodium bicarbonate; and
(b) promptly applying the mixed solution to the oral cavity whereby the carbon dioxide escapes and calcium phosphate is deposited on and around the mucosal tissue of the oral cavity, including but not limited to the cheeks, gums, throat, tongue and other oral soft tissues.

2. The method of claim 1 wherein the resulting solution further contains fluoride ions.

3. The method of claim 1 wherein the pH of the supersaturated solution is from about 5.0 to around 8.0, more preferably from about 6.0-7.5.

4. The method of claim 1 wherein the resulting solution further contains strontium ions.

5. The method of claim 1 wherein the resulting solution further contains chlorhexidine compounds.

6. The method of claim 1 wherein the resulting solution further contains an analgesic such as benzocaine or lidocaine, to provide localized pain relief associated with oral inflammation.

7. The method of claim 1 wherein the inflammatory processes are the result of infections to the mouth, including but not limited to candidiasis, moniliasis, reactivation of latent virus and secondary infections, septicaemia, and combinations thereof.

8. The method of claim 1 for the prevention and treatment of dysphagia and/or stomatitis.

9. The method of claim 1 wherein inflammation is part of the broader syndrome associated with oral mucositis, erythema, Sjogren's Syndrome and any combination thereof

10. The method of claim 1 for treatment of oral inflammation associated with immune-compromised patients including bone marrow transplant patients and AIDS patients.

11. The method of claim 1 for the treatment of side effects of a patient undergoing chemotherapy or radiation treatment.

12. The method of claim 1 wherein other carbonate salts are substituted for, or in addition to sodium bicarbonate, including, but not limited to calcium, potassium or magnesium carbonates.

13. The method of claim 1 wherein solid acids are added to the formulation, including, but not limited to citric or tartaric acids.
